(19) United States
(12) Patent Application Publication
(10) Pub. No.: US 2021/0395665 A1
(43) Pub. Date: Dec. 23, 2021

(54) A Bioreactor System

(71) Applicant: Global Life Sciences Solutions USA LLC, Marlborough, MA (US)

(72) Inventor: Klaus Gebauer, Uppsala (SE)

(21) Appl. No.: 17/281,711

(22) PCT Filed: Oct. 15, 2019
(86) PCT No.: PCT/EP2019/077865

§ 371 Date: Mar. 31, 2021

(30) Foreign Application Priority Data
Oct. 17, 2018 (GB) 1816897.1

(51) Int. Cl. C12M 1/00; C12M 1/12; C12M 1/34; B01D 46/00; B01D 46/44; B01D 46/42

(52) U.S. Cl. CPC C12M 29/20; C12M 37/02; C12M 41/40; C12M 29/04; C12M 29/14; C12M 23/14; B01D 2273/28; C12M 23/28; B01D 46/002; B01D 46/446; B01D 46/4272; B01D 46/0049; B01D 2273/30; C12M 23/26

(57) ABSTRACT

A method for removing exhaust gas from a bioreactor (3) and a bioreactor system. The method comprises the steps of: —providing at least one exhaust filter (15; 115a, 115b) connected to an outlet (7) of the bioreactor (3) for transferring exhaust gas out from the bioreactor; —increasing a pressure at an inlet side (25) of the at least one exhaust filter (15; 115a, 115b) in a connection (16) between the bioreactor (3) and the at least one exhaust filter (15; 115a, 115b) or decreasing a pressure at an outlet side (23) of the at least one exhaust filter (15; 115a, 115b).

### TECHNICAL FIELD OF THE INVENTION

The present invention relates to a method for removing exhaust gas from a bioreactor, a bioreactor system, a control system and an exhaust filter assembly.

### BACKGROUND

A current problem with single-use bioreactors is that that the exhaust gas has to be removed in an efficient manner from the bioreactor using a sterile grade exhaust gas filter. As cell densities are expected to increase in future, more oxygen and gas has to be supplied to the bioreactor and the cell culture, hereby further increasing the volumetric flow and filter sizes in the exhaust. In single-use bioreactors, the strength of the bioreactor bag (the consumable made from flexible film) is typically limited to less than 1 psi (7 kPa). As filters tend to block and foul, i.e. due to moist captured from the humid exhaust gas, a replacement of the filter is required as soon as the pressure inside the bag approaches a certain threshold, for example 0.5 psi (3.5 kPa), in order to not compromise the integrity of the bag and film. As a result, large and/or many filters and corresponding surface areas are required to accommodate for the required gas flow rates, which translates to high cost and physical footprint for the filters at the bioreactor.

### SUMMARY

An object of the present invention is to improve exhaust gas removal in bioreactors.

A further object of the present invention is to improve bioreactor exhaust filter capacity and efficiency.

This is achieved by a method for removing exhaust gas from a bioreactor, a method for extending a life time of an exhaust filter connected to an outlet of a bioreactor for transferring exhaust gas out from the bioreactor, a bioreactor system, a control system and an exhaust filter assembly according to the independent claims.

According to one aspect of the invention a method for removing exhaust gas from a bioreactor is provided. Said method comprises the steps of:
providing at least one exhaust filter connected to an outlet of the bioreactor for transferring exhaust gas out from the bioreactor;
increasing a pressure at an inlet side of the at least one exhaust filter in a connection between the bioreactor and the at least one exhaust filter or decreasing a pressure at an outlet side of the at least one exhaust filter.

According to another aspect of the invention a method for extending a life time of an exhaust filter connected to an outlet of a bioreactor for transferring exhaust gas out from the bioreactor is provided. Said method comprises the step of increasing a pressure at an inlet side of the exhaust filter in a connection between the bioreactor and the exhaust filter or decreasing a pressure at an outlet side of the exhaust filter.

According to another aspect of the invention a bioreactor system is provided comprising:
a bioreactor;
at least one exhaust filter connected to an outlet of the bioreactor for transferring exhaust gas out from the bioreactor; and
a pressure controlling device provided in connection with the at least one exhaust filter and configured for decreasing a pressure at an outlet side of the at least one exhaust filter or increasing a pressure at an inlet side of the at least one exhaust filter.

According to another aspect of the invention a control system configured for being connected to a pressure controlling device in such a bioreactor system is provided.

According to another aspect of the invention an exhaust filter assembly configured for being connected to an outlet of a bioreactor is provided. Said exhaust filter assembly comprises at least one exhaust filter and a pressure controlling device connected to the at least one exhaust filter, which pressure controlling device is configured for decreasing a pressure at an outlet side of the at least one exhaust filter or increasing a pressure at an inlet side of the at least one exhaust filter.

Hereby, by decreasing a pressure at an outlet side of the exhaust filter or increasing a pressure at an inlet side of the exhaust filter the filter capacity is increased. Hereby a smaller exhaust filter can be used for the bioreactor system. This is suitable for cost and space saving reasons.

In one embodiment of the invention the method further comprises the step of providing a pressure controlling device in connection with the at least one exhaust filter, wherein said pressure controlling device is configured for decreasing a pressure at an outlet side of the at least one exhaust filter or increasing a pressure at an inlet side of the at least one exhaust filter.

In one embodiment of the invention the method further comprises the steps of:
measuring a pressure in the bioreactor; and
controlling the pressure controlling device to provide different amount of pressure decrease to the outlet side of the at least one exhaust filter or pressure increase to the inlet side of the at least one exhaust filter in dependence of the measured pressure in the bioreactor.

In this embodiment of the invention the bioreactor system further comprises:
a pressure sensor provided in the bioreactor system for measuring a pressure in the bioreactor; and
a control system, which is connected to the pressure sensor and to the pressure controlling device, wherein said control system is configured for controlling the pressure controlling device to provide different amount of pressure decrease to the outlet side of the at least one exhaust filter or pressure increase to the inlet side of the at least one exhaust filter in dependence of the measured pressure in the bioreactor.

Hereby a pressure in the bioreactor can always be monitored and controlled. In single use applications the bioreactor is often a flexible bag and may not always be supported by a hard support, especially not at the top of the reactor and therefore it is necessary to keep a pressure below a certain limit, for ex 0.5 psi or 1 psi. By adapting the amount of pressure control provided by the pressure controlling device the pressure inside the bioreactor can be kept within predefined limits, for example above atmospheric pressure to avoid collapsing of the flexible bioreactor bag as a lower limit and below an upper limit which is dependent on the strength of the flexible bioreactor bag and/or its components.

In one embodiment of the invention the bioreactor comprises a flexible bioreactor bag.

In one embodiment of the invention said pressure controlling device is a pump and said control system is configured for controlling an effect of the pump in dependence of the measured pressure in the bioreactor.

In one embodiment of the invention the method further comprises providing gas into the bioreactor through an inlet filter. In this embodiment of the invention the bioreactor system further comprises a gas providing device connected to an inlet of the bioreactor arranged for providing gas into the bioreactor.

In one embodiment of the invention the method further comprises:
providing at least a first and a second exhaust filter connected in parallel via at least one valve to an outlet of the bioreactor for transferring exhaust gas out from the bioreactor; and
connecting first the first exhaust filter to the outlet through the at least one valve and when the first exhaust filter is clogged to a certain degree connecting the second exhaust filter to the outlet through the at least one valve.

In this embodiment of the invention the bioreactor system hereby comprises at least a first and a second exhaust filter connected in parallel via at least one valve to an outlet of the bioreactor for transferring exhaust gas out from the bioreactor.

Hereby a first exhaust filter can first be used until it is blocked to a certain degree by moist and then a second exhaust filter can be connected. Hereby a total capacity can be increased and the risk for failure due to a clogged exhaust filter is decreased.

### BRIEF DESCRIPTION OF THE DRAWINGS

FIGS. 1a-1b show two bioreactor systems according to prior art.

FIGS. 2a-2b show two bioreactor systems according to different embodiments of the invention.

FIGS. 3a-3b show two bioreactor systems according to two other embodiments of the invention.

FIG. 4 is a flow chart of a method according to one embodiment of the invention.

### DETAILED DESCRIPTION OF EMBODIMENTS

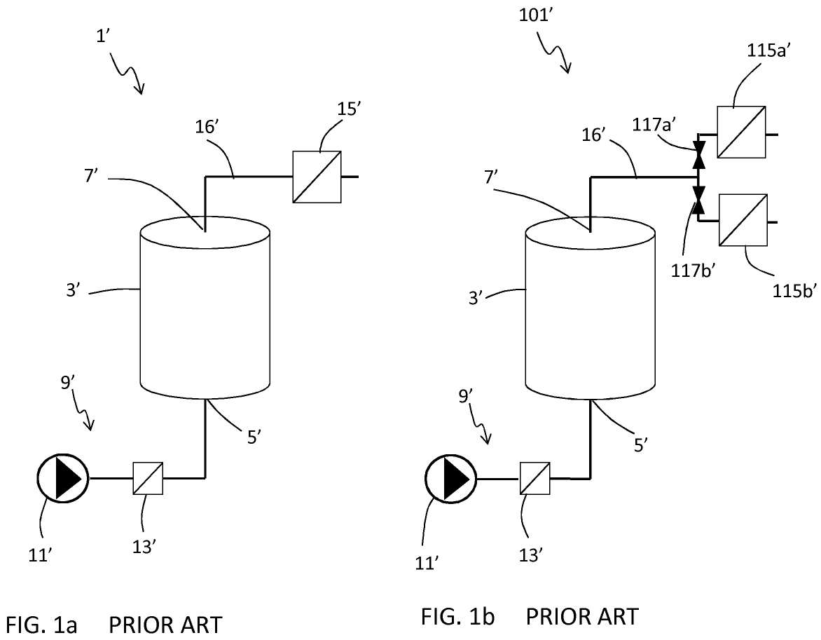

FIG. 1a shows a bioreactor system 1′ according to prior art. It comprises a bioreactor 3′ with an inlet 5′ and an outlet 7′. The bioreactor system 1′ comprises furthermore a gas providing device 9′ which is provided outside the bioreactor 3′ and connected to the inlet 5′ of the bioreactor 3′. The bioreactor system may comprise a sparger submerged into the cell culture fluid (not shown) that is dispersing the gas supplied through inlet 5′. The bioreactor system may further comprise a mixing device, for example an impeller (not shown), which is allowing for homogenization of the cell culture fluid and aiding in the dispersion of the gas, for example for providing good mass transfer of oxygen into the cell culture fluid. Said gas providing device 9′ may comprise a pump 11′ and a sterilizing grade inlet filter 13′ and is arranged for providing gas into the bioreactor 3′. The gas providing device 9′ may further be equipped with a volumetric flow control (not shown), for example in form of a mass flow controller (MFC). The bioreactor system 1′ comprises also an exhaust filter 15′ which is provided outside the bioreactor 3′ and connected to the outlet 7′ of the bioreactor 3′ through a connection 16′ for transferring exhaust gas out from the bioreactor through the exhaust filter 15′. During operation for example moist can get caught in the exhaust filter 15′ which will become more and more blocked as discussed above. In some bioreactor configurations, a condenser for removing moist may be arranged in the gas exhaust line upstream the exhaust filter. Even with a condenser, the fundamental problem of a limited exhaust gas filter capacity remains. In some bioreactor configurations, a heater may be fitted to the exhaust filter to increase the temperature of the filter and thereby reducing the degree of moist trapped in the filter and reducing the filter's capacity. Even with a filter heater, the fundamental problem of a limited exhaust gas filter capacity remains.

For single use applications flexible bags are often used as bioreactors and such flexible bags can only stand a certain degree of pressure. The more the exhaust filter is blocked the higher the pressure inside the bioreactor will be if the gas flow into the bioreactor bag is kept constant. As a result of increasing pressure in the bioreactor bag, either the exhaust filter needs to be changed when it has been blocked too much or additional and thus larger exhaust filter area needs to be provided from the beginning for being able to handle the whole process. Another alternative which has been used in prior art is to provide two or more exhaust filters connected in parallel to the outlet of the bioreactor. This is shown in FIG. 1b. This bioreactor system 101′ is almost identical to the bioreactor system 1′ shown in FIG. 1a except from that two exhaust filters, a first exhaust filter 115a′ and a second exhaust filter 115b′ are provided instead of only one. Furthermore, two valves 117a, 117b are provided in the connection 16′ from the outlet 7′ of the bioreactor 3′ to the two exhaust filters 115a′, 115b′ for providing the possibility to connect either the first exhaust filter 115a′ or the second exhaust filter 115b′ to the bioreactor 3′. Hereby a first exhaust filter 115a′ can first be used until it is blocked to a certain degree by moist and then a second exhaust filter 115b′ can be connected.

Thus, the second, fresh exhaust filter may be either connected and run instead of the first exhaust filter by closing off the first exhaust filter and running only gas through the second filter or the second, fresh exhaust filter may be connected and run in parallel with the first exhaust filter.

As exhaust gas filters may be connected via aseptic connectors, a further possibility is that the arrangement with a second exhaust filter 115b′ is designed as a backup solution, where the second exhaust filter 115b′ is connected on demand using aseptic connectors when the capacity of first filter 115a′ has been exhausted. In other embodiments, three or more exhaust filters may be arranged to further provide capacity and backup capability.

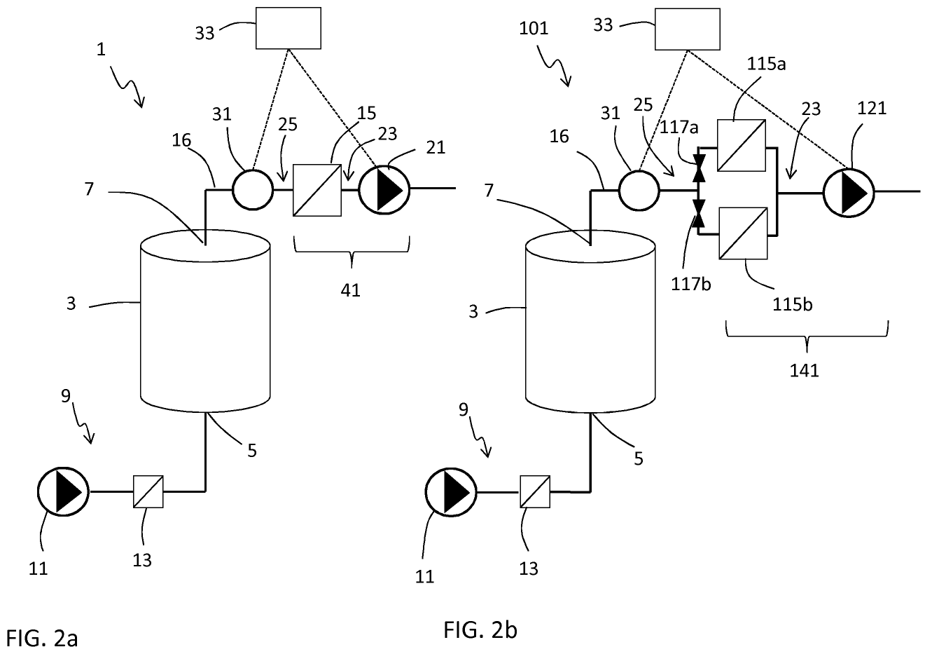

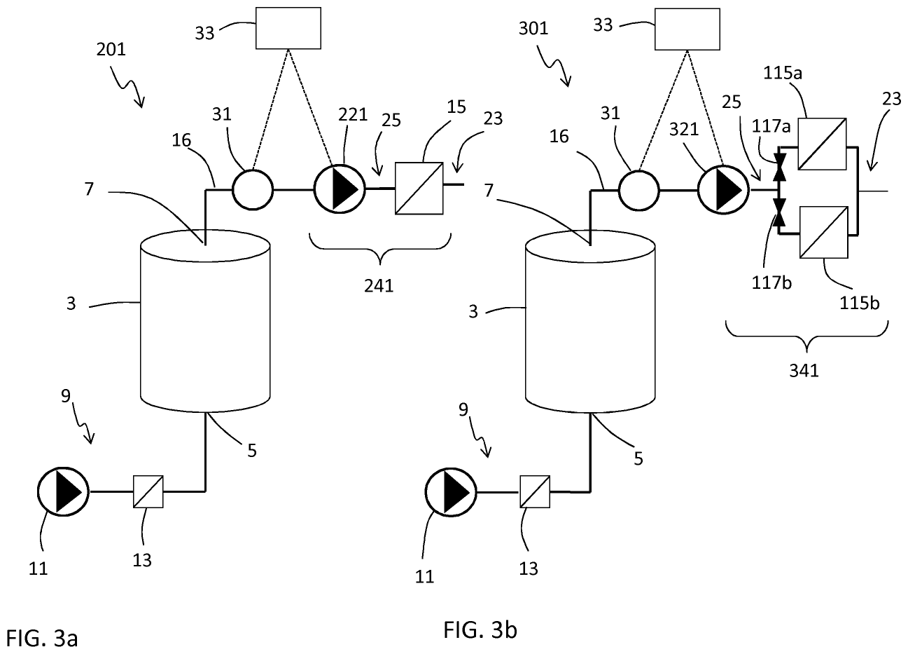

Different embodiments of bioreactor systems 1, 101, 201, 301 according to the invention are described in relation to FIGS. 2a, 2b, 3a and 3b. Many of the components are the same and are also given the same or corresponding reference numbers and will be described together below. The bioreactor system 1, 101, 201, 301 comprises a bioreactor 3 with an inlet 5 and an outlet 7. The bioreactor system 1, 101, 201, 301 comprises furthermore a gas providing device 9 which is provided outside the bioreactor 3 and connected to the inlet 5 of the bioreactor 3. The bioreactor system may comprise a sparger submerged into the cell culture fluid (not shown) that is dispersing the gas supplied through inlet 5. The bioreactor system may further comprise a mixing device, for example an impeller (not shown), which is allowing for homogenization of the cell culture fluid and aiding in the dispersion of the gas, for example for providing good mass transfer of oxygen into the cell culture fluid. Said gas providing device 9 can comprise a pump 11 and an inlet filter 13 and is arranged for providing gas into the bioreactor 3. The bioreactor system 1, 101, 201, 301 comprises also at least one exhaust filter 15, 115a, 115b which is provided outside the bioreactor 3 and connected to the outlet 7 of the bioreactor 3 through a connection 16 for transferring exhaust gas out from the bioreactor through the at least one exhaust filter 15, 115a, 115b. One exhaust filter 15 is provided in the embodiments of FIGS. 2a and 3a and two exhaust filters 115a, 115b are provided in parallel in the embodiments of FIGS. 2b and 3b.

A general description of the inventive concept referring to all the embodiments as shown in FIGS. 2a, 2b, 3a and 3b is first given. According to the invention a pressure controlling device 21, 121, 221, 321 is provided in connection with the at least one exhaust filter 15, 115a, 115b and configured for decreasing a pressure at an outlet side 23 of the at least one exhaust filter or increasing a pressure at an inlet side 25 of the at least one exhaust filter. The assembly of the at least one exhaust filter 15, 115a, 115b and the pressure controlling device 21, 121, 221, 321 are also called an exhaust filter assembly 41, 141, 241, 341. By either decreasing the pressure at the outlet side of the at least one exhaust filter or increasing the pressure at the inlet side of the at least one exhaust filter the capacity and thereby the usage time and life length of the exhaust filter can be increased. Hereby a smaller exhaust filter can be used for the bioreactor system. This is suitable for cost and space saving reasons.

If a pressure controlling device according to the invention is positioned downstream the exhaust filter (FIG. 2), then the pressure at the outlet of the exhaust filter can be decreased below ambient pressure. Hereby, a higher effective pressure differential over the exhaust filter can be realized without exceeding the maximum allowed pressure rating of the bioreactor bag and the pressure at the inlet side of the filter.

If a pressure controlling device according to the invention is positioned upstream the exhaust filter (FIG. 3), then the pressure at the inlet of the exhaust filter can be increased beyond the maximum allowed rating of the bioreactor bag. Hereby, a higher effective pressure differential over the exhaust filter can be realized without exceeding the maximum allowed pressure rating of the bioreactor bag. This arrangement requires of course that the pressure rating of tubing and exhaust filter is higher than the pressure rating of the bioreactor bag upstream the pressure controlling device 221; 321. The arrangement of the pressure controlling device upstream the exhaust filter also requires to provide the pressure controlling device preferably as a pre-sterilized single-use component, either as integrated part of the bioreactor or as a pre-sterilized part that can be connected to the bioreactor at the point of use. An advantage of deploying a pressure controlling device downstream the exhaust filter is that no sterility is required for the device and the device may therefore be a re-usable device that can be re-used over the course of many cell cultures and bioreactor bags, respectively. Thus, providing a pressure controlling device downstream the exhaust filter is preferable for reasons of reduced cost and reduced complexity of the bioreactor as a consumable.

It is understood that pressure ratings for typical exhaust filters, both in regard to maximum allowed pressure and/or maximum allowed pressure differential have to be larger than the maximum pressure allowed for the bioreactor bag and/or the pressure differential between maximum allowed bag pressure and ambient pressure, which is the case with typical exhaust filters. If and where needed to fully exploit the advantages of the proposed invention, adjustments to the design and pressure rating of the components utilized, and in especially the exhaust filters, could be made.

One embodiment of the invention is shown in FIG. 2a. In this embodiment only one exhaust filter 15 is provided and the pressure controlling device 21 is provided in connection with the outlet side 23 of the exhaust filter 15. The pressure controlling device 21 is configured for decreasing the pressure at the outlet side 23 of the exhaust filter 15 compared to the pressure present there without a pressure controlling device 21 provided, which could be for example atmospheric pressure. The pressure controlling device 21 can be for example an air pump that is designed as a centrifugal or fan type pump. Depending on bioreactor size and volumetric air flow, other solutions could be employed, such for example diaphragm type pumps. For large bioreactors and high volumetric gas flows, the fan type pump may be preferable for reasons of cost as to allow simple and robust operation. A fan type pump arrangement may be encapsulated or shielded to avoid turbulent air flow in a clean room environment and exhaust gas flow from the arrangement may be exhausted to an environment external to the processing room and clean room, respectively.

A bioreactor system 101 according to another embodiment of the invention is described in relation to FIG. 2b. In this embodiment the bioreactor system 101 comprises at least a first and a second exhaust filter 115a, 115b connected in parallel via two valves 117a, 117b to an outlet 7 of the bioreactor 3 for transferring exhaust gas out from the bioreactor. The valves 117a, 117b are provided for providing the possibility to connect first one of the exhaust filters and then the other. Also in this embodiment a pressure controlling device 121 is provided in connection with an outlet side 23 of the two parallel exhaust filters 115a, 115b. The pressure controlling device 121 is configured for decreasing the pressure at the outlet side 23 of the exhaust filters 115a, 115b and can be for example a pump or a fan as described above. By using two or more exhaust filters 115a, 115b connected in parallel a filter capacity of the system can be even more increased and the risk for a failure of a process caused by blocked filters can be minimized.

A bioreactor system 201 according to another embodiment of the invention is described in relation to FIG. 3a. In this embodiment the bioreactor system 201 comprises only one exhaust filter 15. Another embodiment of a bioreactor system 301 similar to the one described in relation to FIG. 3a but comprising two exhaust filters 115a, 115b connected in parallel is shown in FIG. 3b. These two embodiments are now described together. A pressure controlling device 221, 321 is according to the invention provided in the bioreactor system 201, 301. In these two embodiments the pressure controlling device 221, 321 is however provided in connection with an inlet side 25 and thus upstream of the at least one exhaust filter 15, 115a, 115b. The pressure controlling device 221, 321 is in these embodiments configured for increasing a pressure at the inlet side 25 of the at least one exhaust filter 15, 115a, 115b. Hereby the capacity of the filter can be increased.

Hereby, as discussed above, the pressure at the inlet of the exhaust filter can be increased beyond the maximum allowed rating of the bioreactor bag. Hereby, a higher effective pressure differential over the exhaust filter can be realized without exceeding the maximum allowed pressure rating of the bioreactor bag. This arrangement requires of course that the pressure rating of tubing and exhaust filter is higher than the pressure rating of the bioreactor bag upstream the pressure increasing device 221; 321.

Common for all the embodiments described above is that the bioreactor system 1, 101, 201, 301 additionally can comprise a pressure sensor 31 provided in the bioreactor system for measuring a pressure in the bioreactor 3. The pressure sensor 31 is shown to be provided in the connection 16 which is connecting the outlet 7 of the bioreactor 3 and the at least one exhaust filter 15, 115a, 115b. However, the pressure sensor 31 can instead be provided in or in another connection to the headspace of the bioreactor bag. The bioreactor headspace is the gas filled volume above the bioreactor's process liquid. When a pressure controlling device is provided upstream and at the inlet of the exhaust filter, an additional pressure sensor (not shown) may be provided in between the pressure controlling device and the inlet of the exhaust filter to monitor and/or control the gas inlet pressure at the inlet of the exhaust filter.

Furthermore, a control system 33 can also be provided in the bioreactor system 1, 101, 201, 301. The control system 33 is connected to the pressure sensor 31 and to the pressure controlling device 21, 121, 221, 321. The control system 33 is configured for controlling the pressure controlling device 21, 121, 221, 321 to provide different amount of pressure decrease to the outlet side 23 of the at least one exhaust filter 15, 115a, 115b or pressure increase to the inlet side 25 of the at least one exhaust filter 15, 115a, 115b in dependence of the measured pressure in the bioreactor 3. Hereby a pressure in the bioreactor 3 can always be monitored and controlled. Preferably, said feedback control is applied to keep the pressure in the bioreactor constant and/or to maintain the exhaust gas flow rate constant, thereby avoiding other variations in the culture process, measurement and control of its parameters and/or to avoid load and stress cycles for the flexible bioreactor bag material.

In single use applications the bioreactor is often a flexible bag and may not always be supported by a hard support, especially not at the top of the reactor and therefore it is necessary to keep a pressure below a certain limit, for ex 0.5 psi or 1 psi. For different systems and different types of bioreactors having different strengths these pressure limits can of course be different.

By adapting the amount of pressure control provided by the pressure controlling device 21, 121, 221, 321 the pressure inside the bioreactor can be kept within predefined limits, for example above atmospheric pressure to avoid collapsing of the flexible bioreactor bag as a lower limit and below an upper limit which is dependent on the strength of the flexible bioreactor bag 3 and/or its components.

The control system 33 can for example be configured for controlling an effect of the pressure controlling device 21, which can be for example a pump, in dependence of the measured pressure in the bioreactor.

In one embodiment of the invention, the effect of the pump is controlled by controlling the speed of the pump. In another embodiment of the invention where a pressure controlling device in the form of a pump is arranged at the outlet of the exhaust filter, a choke type arrangement may be employed for controlling the effect of the pump, where the effect of the pump and the pressure at the exhaust filter is controlled by varying an amount of additional gas flow input to be combined with the gas flow through the exhaust filter, rather than by adjusting the speed of the pump.

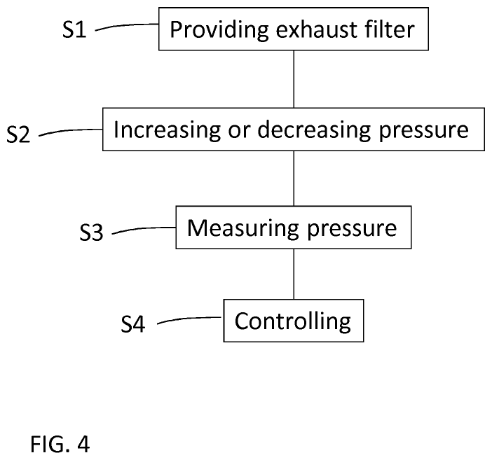

FIG. 4 is a flow chart of a method for removing exhaust gas from a bioreactor according to one embodiment of the invention. The method steps are described in order below:

S1: Providing at least one exhaust filter 15; 115a, 115b connected to an outlet 7 of the bioreactor 3 (which may be a single-use bioreactor, comprising a flexible bag) for transferring exhaust gas out from the bioreactor.

S2: Increasing a pressure at an inlet side 25 of the at least one exhaust filter 15; 115a, 115b in a connection 16 between the bioreactor 3 and the at least one exhaust filter 15; 115a, 115b or decreasing a pressure at an outlet side 23 of the at least one exhaust filter 15; 115a, 115b.

Wherein the method further comprises the step of providing a pressure controlling device 21; 121; 221; 321 (which may be a pump or a fan, preferably a pump) in connection with the at least one exhaust filter 15; 115a, 115b, wherein said pressure controlling device is configured for decreasing a pressure at an outlet side 23 of the at least one exhaust filter 15; 115a, 115b or increasing a pressure at an inlet side 25 of the exhaust filter.

In one embodiment of the invention the method further comprises the steps of:

S3: Measuring a pressure in the bioreactor; and
S4: Controlling the pressure controlling device 21; 121; 221; 321 to provide different amount of pressure decrease to the outlet side 23 of the exhaust filter or pressure increase to the inlet side 25 of the exhaust filter in dependence of the measured pressure in the bioreactor.

The method furthermore comprises providing gas into the bioreactor 3 through an inlet filter 13.

1. A method for removing exhaust gas from a bioreactor, said method comprising the steps of:
providing at least one exhaust filter connected to an outlet of the bioreactor for transferring exhaust gas out from the bioreactor;
increasing a pressure at an inlet side of the at least one exhaust filter in a connection between the bioreactor and the at least one exhaust filter or decreasing a pressure at an outlet side of the at least one exhaust filter.

2. A method for extending a life time of an exhaust filter connected to an outlet of a bioreactor for transferring exhaust gas out from the bioreactor, said method comprising the step of increasing a pressure at an inlet side of the exhaust filter in a connection between the bioreactor and the exhaust filter or decreasing a pressure at an outlet side of the exhaust filter.

3. The method according to claim 1, wherein the method further comprises the step of providing a pressure controlling device in connection with the at least one exhaust filter, wherein said pressure controlling device is configured for decreasing a pressure at an outlet side of the at least one exhaust filter or increasing a pressure at an inlet side of the at least one exhaust filter.

4. The method according to claim 3, wherein the method further comprises the steps of:
measuring a pressure in the bioreactor; and
controlling the pressure controlling device to provide different amount of pressure decrease to the outlet side of the at least one exhaust filter or pressure increase to the inlet side of the at least one exhaust filter; in dependence of the measured pressure in the bioreactor.

5. The method according to claim 1, wherein the bioreactor comprises a flexible bioreactor bag.

6. The method according to claim 5, wherein the bioreactor a single-use bioreactor.

7. The method according to claim 1, wherein the method further comprises providing gas into the bioreactor through an inlet filter.

8. The method according to claim 1, wherein the method further comprises:
providing at least a first and a second exhaust filter connected in parallel via at least one valve to an outlet of the bioreactor for transferring exhaust gas out from the bioreactor; and
connecting first the first exhaust filter to the outlet through the at least one valve and when the first exhaust filter is clogged to a certain degree connecting the second exhaust filter to the outlet through the at least one valve.

9. The method according to claim 1, wherein said pressure controlling device is a pump or a fan, such as a pump.

10. A bioreactor system comprising:
a bioreactor;
at least one exhaust filter connected to an outlet of the bioreactor for transferring exhaust gas out from the bioreactor; and
a pressure controlling device provided in connection with the at least one exhaust filter and configured for decreasing a pressure at an outlet side the at least one exhaust filter or increasing a pressure at an inlet side of the at least one exhaust filter.

11. The bioreactor system according to claim 10, further comprising:
a pressure sensor provided in the bioreactor system measuring a pressure in the bioreactor; and
a control system, which is connected to the pressure sensor and to the pressure controlling device, wherein said control system is configured for controlling the pressure controlling device to provide different amount of pressure decrease to the outlet side of the at least one exhaust filter or pressure increase to the inlet side of the at least one exhaust filter in dependence of the measured pressure in the bioreactor.

12. The bioreactor system according to claim 10, wherein said pressure controlling device is a pump or a fan, such as a pump.

13. The bioreactor system according to claim 11, wherein said control system configured for controlling an effect of the pump in dependence of the measured pressure in the bioreactor.

14. The bioreactor system according to claim 10, wherein said pressure controlling device is provided in connection with the outlet side of the at least one exhaust filter and is configured for decreasing the pressure at the outlet side of the at least one exhaust filter.

15. The bioreactor system according to claim 10, wherein the bioreactor system further comprises a gas providing device connected to an inlet of the bioreactor arranged for providing gas into the bioreactor.

16. The bioreactor system according to claim 10, wherein the bioreactor comprises a flexible bioreactor bag.

17. The bioreactor system according to claim 16, wherein the bioreactor is a single-use bioreactor.

18. The bioreactor system according to claim 10, wherein the bioreactor system comprises at least a first and a second exhaust filter connected in parallel via at least one valve to an outlet of the bioreactor for transferring exhaust gas out from the bioreactor.

19. A control system configured for being connected to a pressure controlling device in the bioreactor system according to claim 8, wherein said control system further is configured for being connected to a pressure sensor provided in the bioreactor system for measuring a pressure in the bioreactor and wherein said control system comprises software for controlling the pressure controlling device to provide different amount of pressure decrease to the outlet side of the at least one exhaust filter or pressure increase to the inlet side of the at least one exhaust filter in dependence of the pressure measured by the pressure sensor.

20. An exhaust filter assembly configured for being connected to an outlet of a bioreactor, wherein said exhaust filter assembly comprises at least one exhaust filter and a pressure controlling device connected to the at least one exhaust filter, which pressure controlling device is configured for decreasing a pressure at an outlet side of the at least one exhaust filter or increasing a pressure at an inlet side of the at least one exhaust filter.

21. The exhaust filter assembly according to claim 20, wherein said pressure controlling device is configured to be controllable to provide different amount of pressure decrease to the outlet side of the at least one exhaust filter or pressure increase to the inlet side of the at least one exhaust filter.

22. The exhaust filter assembly according to claim 20, wherein said pressure controlling device is a pump or a fan, such as a pump.

23. The exhaust filter assembly according to claim 20, wherein said pressure controlling device is provided in connection with an outlet side from the at least one exhaust filter and is configured for decreasing the pressure at the outlet side of the at least one exhaust filter.
